(19) United States
(12) Patent Application Publication
(10) Pub. No.: US 2026/0009089 A1
(43) Pub. Date: Jan. 8, 2026

(54) SINGLE NUCLEOTIDE POLYMORPHISM (SNP) MOLECULAR MARKER COMBINATION ASSOCIATED WITH VERTICILLIUM WILT RESISTANCE IN UPLAND COTTON AND USE THEREOF

(71) Applicant: HUAZHONG AGRICULTURAL UNIVERSITY, Wuhan, Hubei (CN)

(72) Inventors: Longfu ZHU, Wuhan, Hubei (CN); Xiaojun ZHANG, Wuhan, Hubei (CN); Shiming LIU, Wuhan, Hubei (CN); Shuangxia JIN, Wuhan, Hubei (CN); Xianlong ZHANG, Wuhan, Hubei (CN)

(21) Appl. No.: 19/117,268

(22) PCT Filed: Oct. 9, 2024
(86) PCT No.: PCT/CN2024/123543

§ 371 Date: Mar. 31, 2025

(30) Foreign Application Priority Data
Oct. 13, 2023 (CN) 202311322179.8

(51) Int. Cl. C12Q 1/6895

(52) U.S. Cl. CPC C12Q 1/6895; C12Q 2600/13; C12Q 2600/156

(57) ABSTRACT

A single nucleotide polymorphism (SNP) molecular marker associated with Verticillium wilt resistance in upland cotton is provided, including 10 SNP molecular markers, where specific SNP sites and polymorphisms of the SNP molecular markers are also given. Based on the 10 SNP molecular markers, two primer sets are provided for application: one primer set (10 pairs) of sequencing primers for amplifying a DNA fragment where the SNP is located, combined with barcode, can obtain resistant/susceptible genotype information of multiple samples in high throughput; the other primer set (10 pairs) of typing primers for ordinary PCR is provided, and can differentiate resistant/susceptible genotypes by an electrophoresis band type. The combination of the 10 SNP molecular markers associated with Verticillium wilt resistance can be used for early prediction and screening of the Verticillium wilt resistance in the upland cotton, thereby assisting in the improvement of disease resistance traits for cotton varieties.

### CROSS-REFERENCE TO RELATED APPLICATION

The present application is a national stage application of International Patent Application No: PCT/CN2024/123543 filed on Oct. 9, 2024 and claims priority to the Chinese Patent Application No. 2023113221798, filed with the China National Intellectual Property Administration (CNIPA) on Oct. 13, 2023, and entitled “SINGLE NUCLEOTIDE POLYMORPHISM (SNP) MOLECULAR MARKER ASSOCIATED WITH VERTICILLIUM WILT RESISTANCE IN UPLAND COTTON AND USE THEREOF”, which is incorporated herein by reference in its entirety.

### REFERENCE TO SEQUENCE LISTING

A computer readable XML file entitled “GWPCTP20241207898_sequence listing”, which was created on Dec. 24, 2024, with a file size of about 58,404 bytes, contains the sequence listing for this application, has been filed with this application, and is hereby incorporated by reference in its entirety.

### TECHNICAL FIELD

The present disclosure belongs to the technical field of biological agriculture, and specifically relates to a single nucleotide polymorphism (SNP) molecular marker associated with Verticillium wilt resistance in upland cotton among plant molecular markers and use thereof.

### BACKGROUND

Existing studies have shown that the resistance to Verticillium wilt in cotton is a complex quantitative inheritance, which is controlled by multiple genes and easily affected by the environment. In recent years, researchers have located not less than 200 quantitative trait loci (QTLs) for Verticillium wilt resistance in upland cotton by linkage analysis using different genetic populations, but so far there has been no report on the cloning of major resistance QTLs or resistance genes. Most of the genes involved in Verticillium wilt resistance that have been identified are also located downstream of the disease resistance pathway and show weak effects. It is difficult to fully understand the disease resistance mechanism through the study of a single gene, and the molecular marker-assisted disease resistance breeding developed using a single QTL also has low selection efficiency and unsatisfactory results. Therefore, the modern molecular breeding era faces complex traits such as cotton Verticillium wilt that are significantly affected by the environment. In order to identify QTL with a broad spectrum of disease resistance and strong effects in a field environment with complex fungal strains, it is necessary to conduct multi-year and multi-point trait investigations on genetic populations to identify a number of stable and key QTL. The multiple sites that appear stably in multiple environments combine the pressures of different regions, temperature and humidity conditions, soil environment, and multiple dominant fungal strains. By using such a group of QTL to develop a set of molecular marker combinations and conducting genomic selection breeding by pyramiding resistant alleles, the disease resistance of cotton varieties can be comprehensively enhanced, the innovation of cotton germplasm resistant to Verticillium wilt can be accelerated, and breeding results can be improved.

### SUMMARY

The present disclosure provides a combination of single nucleotide polymorphism (SNP) molecular markers associated with Verticillium wilt resistance in upland cotton, including 10 SNP molecular markers from Lsnp1 to Lsnp10; where

the SNP molecular marker Lsnp1 has the nucleotide sequence shown in SEQ ID NO: 1, a polymorphic site located at position 66 of the sequence shown in SEQ ID NO: 1, and a polymorphism of A/G;

the SNP molecular marker Lsnp2 has the nucleotide sequence shown in SEQ ID NO: 2, a polymorphic site located at position 144 of the sequence shown in SEQ ID NO: 2, and a polymorphism of C/T;

the SNP molecular marker Lsnp3 has the nucleotide sequence shown in SEQ ID NO: 3, a polymorphic site located at position 67 of the sequence shown in SEQ ID NO: 3, and a polymorphism of T/A;

the SNP molecular marker Lsnp4 has the nucleotide sequence shown in SEQ ID NO: 4, a polymorphic site located at position 127 of the sequence shown in SEQ ID NO: 4, and a polymorphism of C/T;

the SNP molecular marker Lsnp5 has the nucleotide sequence shown in SEQ ID NO: 5, a polymorphic site located at position 126 of the sequence shown in SEQ ID NO: 5, and a polymorphism of T/C;

the SNP molecular marker Lsnp6 has the nucleotide sequence shown in SEQ ID NO: 6, a polymorphic site located at position 125 of the sequence shown in SEQ ID NO: 6, and a polymorphism of T/A;

the SNP molecular marker Lsnp7 has the nucleotide sequence shown in SEQ ID NO: 7, a polymorphic site located at position 112 of the sequence shown in SEQ ID NO: 7, and a polymorphism of T/A;

the SNP molecular marker Lsnp8 has the nucleotide sequence shown in SEQ ID NO: 8, a polymorphic site located at position 112 of the sequence shown in SEQ ID NO: 8, and a polymorphism of T/C;

the SNP molecular marker Lsnp9 has the nucleotide sequence shown in SEQ ID NO: 9, a polymorphic site located at position 93 of the sequence shown in SEQ ID NO: 9, and a polymorphism of C/T; and

the SNP molecular marker Lsnp10 has the nucleotide sequence shown in SEQ ID NO: 10, a polymorphic site located at position 90 of the sequence shown in SEQ ID NO: 10, and a polymorphism of T/C.

Further, disease resistance dominant allele types of the SNP molecular markers Lsnp1to Lsnp10 are AA, CC, TT, CC, TT, TT, TT, TT, CC, and TT in sequence.

The present disclosure further provides a method for detecting Verticillium wilt resistance in upland cotton, including: determining disease resistance of the upland cotton by detecting genotypes of the 10 SNP molecular markers; where

physical location information of the 10 SNP molecular markers is as follows:

| QTL name | SNP name | Chromosome | Physical location | SNP type |
|---|---|---|---|---|
| qVWR.A01.1 | Lsnp1 | A01 | 111929609 | A/G |
| qVWR.A01.2 | Lsnp2 | A01 | 117983536 | C/T |
| qVWR.A07.2 | Lsnp3 | A07 | 90971603 | T/A |
| qVWR.A10.1 | Lsnp4 | A10 | 108893925 | C/T |
| qVWR.A11.2 | Lsnp5 | A11 | 119799615 | T/C |
| qVWR.A13.1 | Lsnp6 | A13 | 105434827 | T/A |
| qVWR.D01.1 | Lsnp7 | D01 | 1696495 | T/A |
| qVWR.D07.1 | Lsnp8 | D07 | 13371656 | T/C |
| qVWR.D08.2 | Lsnp9 | D08 | 61445110 | C/T |
| qVWR.D10.1 | Lsnp10 | D10 | 22542365 | T/C |

the physical location information of the SNP molecular markers is determined based on a genome of Gossypium hirsutum (AD1) ‘TM-1’ genome ZJU-improved_v2.1_a1 version in a standard line Texas Marker-1 of the upland cotton.

Further, a criterion for detecting the Verticillium wilt resistance in the upland cotton is that larger number of resistant genotypes indicates a stronger disease resistance.

The present disclosure further provides primer sequences for typing the 10 SNP molecular markers Lsnp1 to Lsnp10, and the primer sequences for genotyping are shown in SEQ ID NO: 11 to SEQ ID NO: 30 in sequence.

The present disclosure further provides use of the SNP molecular marker or the primer set in early prediction, screening, or breeding of Verticillium wilt resistance in upland cotton.

The present disclosure further provides a primer set for high-throughput detection of Verticillium wilt resistance in upland cotton, where the primer set is used for amplifying nucleotide fragments where the 10 SNP molecular markers (Lsnp1 to Lsnp10) are located, and primer sequences corresponding to the 10 SNP molecular markers Lsnp1 to Lsnp10 are shown in SEQ ID NO: 31 to SEQ ID NO: 50 in sequence.

Specifically, a PCR amplification result of a primer set having the primer sequences shown in SEQ ID NO: 31 to SEQ ID NO: 32 shows positive, indicating a susceptible genotype, and an annealing temperature is 54° C. to 57° C.;

a PCR amplification result of a primer set having the primer sequences shown in SEQ ID NO: 33 to SEQ ID NO: 34 shows positive, indicating a susceptible genotype, and an annealing temperature is 50° C. to 55° C.;

a PCR amplification result of a primer set having the primer sequences shown in SEQ ID NO: 35 to SEQ ID NO: 36 shows positive, indicating a resistant genotype, and an annealing temperature is 65° C. to 67° C.;

a PCR amplification result of a primer set having the primer sequences shown in SEQ ID NO: 37 to SEQ ID NO: 38 shows positive, indicating a resistant genotype, and an annealing temperature is 50° C. to 52° C.;

a PCR amplification result of a primer set having the primer sequences shown in SEQ ID NO: 39 to SEQ ID NO: 40 shows positive, indicating a resistant genotype, and an annealing temperature is 50° C. to 55° C.;

a PCR amplification result of a primer set having the primer sequences shown in SEQ ID NO: 41 to SEQ ID NO: 42 shows positive, indicating a susceptible genotype, and an annealing temperature is 50° C. to 55° C.;

a PCR amplification result of a primer set having the primer sequences shown in SEQ ID NO: 43 to SEQ ID NO: 44 shows positive, indicating a resistant genotype, and an annealing temperature is 50° C. to 53.5° C.;

a PCR amplification result of a primer set having the primer sequences shown in SEQ ID NO: 45 to SEQ ID NO: 46 shows positive, indicating a resistant genotype, and an annealing temperature is 55° C. to 57° C.;

a PCR amplification result of a primer set having the primer sequences shown in SEQ ID NO: 47 to SEQ ID NO: 48 shows positive, indicating a susceptible genotype, and an annealing temperature is 58° C. to 60° C.; and

a PCR amplification result of a primer set having the primer sequences shown in SEQ ID NO: 49 to SEQ ID NO: 50 shows positive, indicating a resistant genotype, and an annealing temperature is 55° C. to 58° C.

The present disclosure further provides use of the primer set in early prediction, screening, or breeding of Verticillium wilt resistance in upland cotton.

The present disclosure further provides a kit for detecting Verticillium wilt resistance in upland cotton, including the primer set.

The present disclosure has the following beneficial effects:

(1) In the present disclosure, 20 groups of phenotypic values are obtained by planting 290 natural cotton populations in three natural disease nurseries in Xinjiang for three years and then conducting a survey on resistance to Verticillium wilt. The association analysis of 20 sets of disease indexs combined with high-density SNP markers covering the whole genome locate 10 stable QTLs in total that can be repeatedly detected in three or more analyses. The most significant SNP loci among the 10 stable QTLs are recorded as Lead-SNPs, and the association between different haplotypes of the 10 Lead-SNPs and disease index differences all reach an extremely significant level (FIG. 2). The number of resistant genotypes carried by cotton materials at the 10 Lsnp sites is positively associated with disease resistance (FIG. 3). Prior to the present disclosure, there was no technique for providing a set of key sites for the complex quantitative inheritance of a Verticillium wilt resistance trait in cotton. The present disclosure is beneficial for comprehensively and effectively improving the disease resistance of cotton varieties.

(2) In the present disclosure, two sets of primers are provided for application based on the 10 Lsnp: one primer set (10 pairs) of typing primers for common PCR and the other primer set (10 pairs) of sequencing primers for amplifying a DNA fragment where the SNP is located. The primer sets can be used to detect the genotype information of single and multiple samples at 10 Lsnp sites, respectively. The molecular marker and primer set can be used for early prediction and evaluation of Verticillium wilt resistance in cotton, and can assist in breeding cotton varieties resistant to Verticillium wilt. Moreover, the use is a result at the DNA level, and the detection does not need to consider the growth period and tissue type of cotton, nor does it need to conduct inoculation and disease resistance identification experiments. As a result, the SNP molecular marker and primer set are not restricted by pathogens, temperature, and humidity, and can accurately and quickly obtain genotype information of single and multiple samples, which is beneficial to accelerate the innovation of cotton germplasm resistant to Verticillium wilt and improve breeding results.

### BRIEF DESCRIPTION OF THE DRAWINGS

FIG. 1 shows a heat map of multi-year multi-point association analysis of Verticillium wilt resistance phenotypes of 290 natural populations in upland cotton;

FIG. 2 shows a box plot of haplotype analysis on disease resistance advantage of 10 Lsnp sites;

FIG. 3A-FIG. 3B show a box plot of pyramiding effect of 10 resistant genotypes among the Upland cotton accessions, where the ordinate represents the disease index and the abscissa represents the number of resistant genotypes carried among the 10 Lsnp sites;

FIG. 4 shows that frequency of 10 resistant genotypes in the Chinese descendible cultivars;

FIG. 5 shows density plots of 272 F2 individuals were determined based on high-throughput sequencing of 5 Lsnp, and the genotype of each material was determined based on the proportions of the three peaks corresponding to the horizontal axis;

FIG. 6 shows the results of typing 272 individual strains at Lsnp4, Lsnp5, Lsnp7, Lsnp8, and Lsnp10 according to the peak patterns in FIG. 5; and

FIG. 7 shows a schematic diagram of electrophoresis for amplifying specific DNA fragments between resistant and susceptible materials using a typing primer set designed based on the 10 Lsnp sites.

### DETAILED DESCRIPTION OF THE EMBODIMENTS

In the present disclosure, 20 groups of phenotypic values are obtained by planting 290 natural cotton populations in three natural disease nurseries in Xinjiang for three years and then conducting a survey on resistance to Verticillium wilt. An association analysis is conducted on 20 groups of phenotypic values using high-density SNP markers covering the whole genome, and a total of 10 SNP markers are located that are associated with the Verticillium wilt resistance trait in cotton. The 290 upland cotton cultivars used are selected from upland cotton cultivars with genome re-sequencing provided by the National Key Laboratory of Crop Genetic Improvement, Huazhong Agricultural University, with reference to “Wang et al. Asymmetric subgenome selection and cis-regulatory divergence during cotton domestication. Nat Genet, 2017, 49:579-587”.

The present disclosure provides a combination of 10 SNP molecular markers Lsnp1 to Lsnp10 associated with Verticillium wilt resistance in upland cotton:

the SNP molecular marker Lsnp1 has the nucleotide sequence shown in SEQ ID NO: 1, a polymorphic site located at position 66 of the sequence shown in SEQ ID NO: 1, and a polymorphism of A/G;

the SNP molecular marker Lsnp2 has the nucleotide sequence shown in SEQ ID NO: 2, a polymorphic site located at position 144 of the sequence shown in SEQ ID NO: 2, and a polymorphism of C/T;

the SNP molecular marker Lsnp3 has the nucleotide sequence shown in SEQ ID NO: 3, a polymorphic site located at position 67 of the sequence shown in SEQ ID NO: 3, and a polymorphism of T/A;

the SNP molecular marker Lsnp4 has the nucleotide sequence shown in SEQ ID NO: 4, a polymorphic site located at position 127 of the sequence shown in SEQ ID NO: 4, and a polymorphism of C/T;

the SNP molecular marker Lsnp5 has the nucleotide sequence shown in SEQ ID NO: 5, a polymorphic site located at position 126 of the sequence shown in SEQ ID NO: 5, and a polymorphism of T/C;

the SNP molecular marker Lsnp6 has the nucleotide sequence shown in SEQ ID NO: 6, a polymorphic site located at position 125 of the sequence shown in SEQ ID NO: 6, and a polymorphism of T/A;

the SNP molecular marker Lsnp7 has the nucleotide sequence shown in SEQ ID NO: 7, a polymorphic site located at position 112 of the sequence shown in SEQ ID NO: 7, and a polymorphism of T/A;

the SNP molecular marker Lsnp8 has the nucleotide sequence shown in SEQ ID NO: 8, a polymorphic site located at position 112 of the sequence shown in SEQ ID NO: 8, and a polymorphism of T/C;

the SNP molecular marker Lsnp9 has the nucleotide sequence shown in SEQ ID NO: 9, a polymorphic site located at position 93 of the sequence shown in SEQ ID NO: 9, and a polymorphism of C/T; and

the SNP molecular marker Lsnp10 has the nucleotide sequence shown in SEQ ID NO: 10, a polymorphic site located at position 90 of the sequence shown in SEQ ID NO: 10, and a polymorphism of T/C.

Further, disease resistance dominant allele types of the SNP molecular markers Lsnp1 to Lsnp10 are AA, CC, TT, CC, TT, TT, TT, TT, CC, and TT in sequence.

The present disclosure further provides a method for detecting Verticillium wilt resistance in upland cotton, including: determining disease resistance of the upland cotton by detecting genotypes of the SNP molecular markers Lsnp1 to Lsnp10; where

physical location information of the 10 SNP molecular markers is as follows:

| QTL name | SNP name | Chromosome | Physical location | SNP type |
|---|---|---|---|---|
| qVWR.A01.1 | Lsnp1 | A01 | 111929609 | A/G |
| qVWR.A01.2 | Lsnp2 | A01 | 117983536 | C/T |
| qVWR.A07.2 | Lsnp3 | A07 | 90971603 | T/A |
| qVWR.A10.1 | Lsnp4 | A10 | 108893925 | C/T |
| qVWR.A11.2 | Lsnp5 | A11 | 119799615 | T/C |
| qVWR.A13.1 | Lsnp6 | A13 | 105434827 | T/A |
| qVWR.D01.1 | Lsnp7 | D01 | 1696495 | T/A |
| qVWR.D07.1 | Lsnp8 | D07 | 13371656 | T/C |
| qVWR.D08.2 | Lsnp9 | D08 | 61445110 | C/T |
| qVWR.D10.1 | Lsnp10 | D10 | 22542365 | T/C |

the physical location information of the SNP molecular markers is determined based on a genome of Gossypium hirsutum (AD1) ‘TM-1’ genome ZJU-improved_v2.1_a1 version in a standard line Texas Marker-1 of the upland cotton.

Further, a criterion for detecting the Verticillium wilt resistance in the upland cotton is that larger number of resistant genotypes indicates a stronger disease resistance.

The present disclosure further provides a primer set for amplifying nucleotide sequences of the 10 SNP molecular markers, which are shown in SEQ ID NO: 11 to SEQ ID NO: 30 in sequence.

Preferably, the primer set is used as a sequencing primer set in combination with a barcode to obtain the resistant/susceptible genotype information of multiple samples in a high-throughput manner.

Preferably, the method for detecting Verticillium wilt resistance in upland cotton includes: quickly acquiring resistant/susceptible genotype information of multiple samples through a barcode and next-generation sequencing. The specific steps are as follows:

(1) extracting genomic DNA of a sample to be tested;

(2) designing the barcode and an adapter according to the demands of multiple samples, adding the adapter to allow synthesis based on the primer series shown in SEQ ID NO: 11 to SEQ ID NO: 30 as needed, conducting PCR on the multiple samples, and then mixing and purifying obtained products; and

(3) conducting the next-generation sequencing, extracting multiple sample information according to the barcode, and analyzing genotypes of each cotton sample at 10 Lsnp sites to analyze disease resistance to the Verticillium wilt.

The present disclosure further provides use of the 10 SNP molecular markers in early prediction, screening, or breeding of Verticillium wilt resistance in upland cotton.

Further, the present disclosure further provides a primer set for detecting Verticillium wilt resistance in upland cotton, where the primer set is used for amplifying nucleotide fragments where the SNP molecular markers (Lsnp1 to Lsnp10) are located, and primer sequences corresponding to the 10 SNP molecular markers Lsnp1 to Lsnp10 are shown in SEQ ID NO: 31 to SEQ ID NO: 50 in sequence.

Preferably, the primer set as a PCR typing primer set can specifically amplify nucleotide fragments where the 10 SNP molecular markers (Lsnp1 to Lsnp10) are located in resistant or susceptible materials.

Specifically, a PCR amplification result of a primer set having the primer sequences shown in SEQ ID NO: 31 to SEQ ID NO: 32 shows positive, indicating a susceptible genotype, and an annealing temperature is 54° C. to 57° C.;

a PCR amplification result of a primer set having the primer sequences shown in SEQ ID NO: 33 to SEQ ID NO: 34 shows positive, indicating a susceptible genotype, and an annealing temperature is 50° C. to 55° C.;

a PCR amplification result of a primer set having the primer sequences shown in SEQ ID NO: 35 to SEQ ID NO: 36 shows positive, indicating a resistant genotype, and an annealing temperature is 65° C. to 67° C.;

a PCR amplification result of a primer set having the primer sequences shown in SEQ ID NO: 37 to SEQ ID NO: 38 shows positive, indicating a resistant genotype, and an annealing temperature is 50° C. to 52° C.;

a PCR amplification result of a primer set having the primer sequences shown in SEQ ID NO: 39 to SEQ ID NO: 40 shows positive, indicating a resistant genotype, and an annealing temperature is 50° C. to 55° C.;

a PCR amplification result of a primer set having the primer sequences shown in SEQ ID NO: 41 to SEQ ID NO: 42 shows positive, indicating a susceptible genotype, and an annealing temperature is 50° C. to 55° C.;

a PCR amplification result of a primer set having the primer sequences shown in SEQ ID NO: 43 to SEQ ID NO: 44 shows positive, indicating a resistant genotype, and an annealing temperature is 50° C. to 53.5° C.;

a PCR amplification result of a primer set having the primer sequences shown in SEQ ID NO: 45 to SEQ ID NO: 46 shows positive, indicating a resistant genotype, and an annealing temperature is 55° C. to 57° C.;

a PCR amplification result of a primer set having the primer sequences shown in SEQ ID NO: 47 to SEQ ID NO: 48 shows positive, indicating a susceptible genotype, and an annealing temperature is 58° C. to 60° C.; and

a PCR amplification result of a primer set having the primer sequences shown in SEQ ID NO: 49 to SEQ ID NO: 50 shows positive, indicating a resistant genotype, and an annealing temperature is 55° C. to 58° C.

Further provided is the use of the primer set in early prediction, screening, or breeding of Verticillium wilt resistance in upland cotton.

The present disclosure further provides a kit for detecting Verticillium wilt resistance in upland cotton, including one of the primer set combinations.

In the present disclosure, unless otherwise specified, all raw material components are commercially available products well known to persons skilled in the art. The technical solutions in the present disclosure are clearly and completely described below with reference to the examples of the present disclosure. Apparently, the described examples are merely a part rather than all of the examples of the present disclosure. All other examples obtained by those skilled in the art based on the examples of the present disclosure without creative efforts shall fall within the protection scope of the present disclosure.

### Example 1

Acquisition of SNP molecular markers associated with Verticillium wilt resistance in upland cotton:

(1) Planting and resistant phenotype investigation of natural cotton population materials:

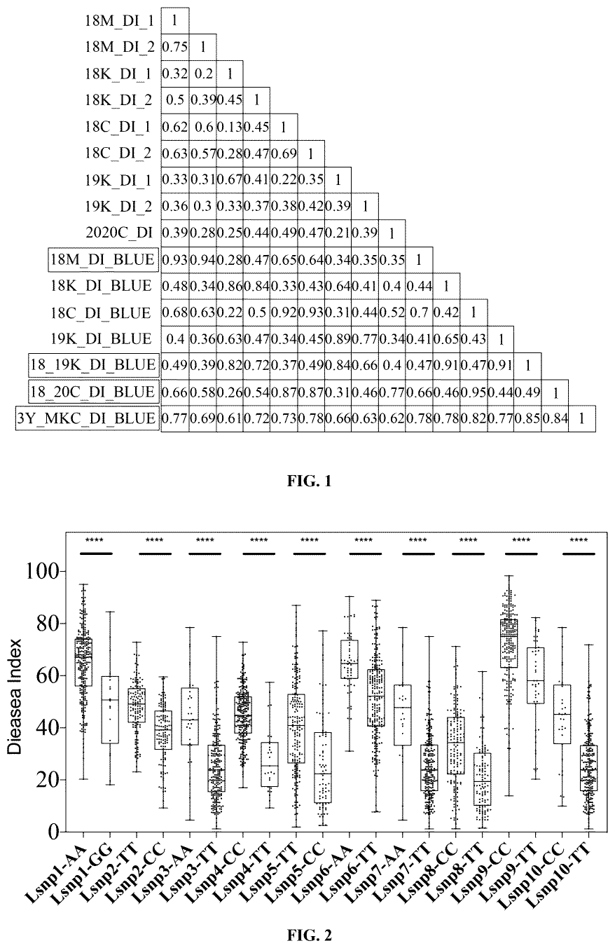

The 290 upland cotton cultivars used were selected from upland cotton cultivars with genome re-sequencing provided by the National Key Laboratory of Crop Genetic Improvement, Huazhong Agricultural University, with reference to “Wang et al. Asymmetric subgenome selection and cis-regulatory divergence during cotton domestication. Nat Genet, 2017, 49:579-587”. From 2018 to 2020, the natural population was planted in three natural disease nurseries of Verticillium wilt in Manas, Korla, and Kuche in Xinjiang. After the disease occurred in July and August, the disease index survey is conducted, and the disease index data of 1 year, 2 years, and 2 years are collected in the three places, respectively. Except for Kuche in 2020, which had only one replicate, there were two replicates in the other years. According to the analysis of the manually counted disease index, the frequency distribution of the disease index showed a normal distribution and was consistent with the genetic characteristics of quantitative traits. The association analysis of multi-environment disease indexes using the Pearson coefficient (R²) proved that the R² (0.33-0.67) at the same location was higher than the R² (0.13-0.63) in the same year, indicating that there was higher stability at the same location. As shown in FIG. 1: the figure showed the heat map of the association analysis of the Verticillium wilt resistance phenotypes of 290 natural populations of upland cotton at three points in three years. The values were the Pearson coefficient calculated between two varibales. The correlation coefficient ranges from 0 to 1, indicating the degree of positive correlation. The phenotype included 9 groups of raw phenotypic values, which were numbered in the format of year location_DI_repeat (18M_DI_1 represented the first repeated disease index in Manas in 2018). The results showed that there was closer association between different years in the same location. After best linear unbiased estimate (BLUE) treatment with location as a fixed factor, 7groups of treatment phenotypic values were obtained, numbered in the format of year location_DI_BLUE (18_19K_DI_BLUE represented the disease index after BLUE treatment of the four groups of phenotypes in Korla in 2018 and 2019).

By using the fixed-effect BLUE to process the disease indicators with repeated locations and materials, the R² of the same location was improved (0.47-0.66). In addition, all the phenotypic data of three points in three years were processed to obtain a comprehensive phenotypic value, and the association coefficient with each phenotype ranged at 0.61-0.85. Thus, 9 groups of original phenotypic values were obtained in three years and three locations, and 7 groups of treated phenotypic values were obtained according to BLUE treatment. In addition, based on practical experience, in order to reduce the error of uneven pathogen content in natural disease gardens, repeated data were combined according to location to obtain 5 groups of combined phenotypic values, totaling 20 groups of phenotypic values.

(2) Genome-wide association study (GWAS) of Verticillium wilt resistance in cotton:

According to the 290 upland cotton cultivars whose genomes had been re-sequenced provided by the National Key Laboratory of Crop Genetic Improvement, Huazhong Agricultural University, and the re-sequencing data were downloaded for population SNP detection. The sequencing data were aligned to the genome Gossypium hirsutum (AD1) ‘TM-1’ genome ZJU-improved_v2.1_a1 of an upland cotton standard line Texas Marker-1 using BWA (0.7.17). After the removal of duplicate sequences and the back-alignment, GATK was used to generate gvcf files for each sample using GATK GenotypeGVCFs. GATK CombineGVCF was used to merge the gvef files of all samples and generate a total vef file. VCFtools was used to filter SNPs, with the criteria being DP (depth, sequencing depth>5), missing rate (missing>50), and minimum allele frequency (MAF>0.05). The final retained vcf was used for association analysis using GEMMA. The 20 groups of phenotypic values in step (1) were subjected to GWAS, and 10 stable QTLs that could be repeatedly detected in three or more analyses were located in total. The most significant SNP sites among the 10 stable QTLs were recorded as Lead-SNPs, named Lsnp1 to Lsnp10, with sequence information as follows:

the SNP molecular marker Lsnp1 had the nucleotide sequence shown in SEQ ID NO: 1, a polymorphic site located at position 66 of the sequence shown in SEQ ID NO: 1, and a polymorphism of A/G;

the SNP molecular marker Lsnp2 had the nucleotide sequence shown in SEQ ID NO: 2, a polymorphic site located at position 144 of the sequence shown in SEQ ID NO: 2, and a polymorphism of C/T;

the SNP molecular marker Lsnp3 had the nucleotide sequence shown in SEQ ID NO: 3, a polymorphic site located at position 67 of the sequence shown in SEQ ID NO: 3, and a polymorphism of T/A;

the SNP molecular marker Lsnp4 had the nucleotide sequence shown in SEQ ID NO: 4, a polymorphic site located at position 127 of the sequence shown in SEQ ID NO: 4, and a polymorphism of C/T;

the SNP molecular marker Lsnp5 had the nucleotide sequence shown in SEQ ID NO: 5, a polymorphic site located at position 126 of the sequence shown in SEQ ID NO: 5, and a polymorphism of T/C;

the SNP molecular marker Lsnp6 had the nucleotide sequence shown in SEQ ID NO: 6, a polymorphic site located at position 125 of the sequence shown in SEQ ID NO: 6, and a polymorphism of T/A;

the SNP molecular marker Lsnp7 had the nucleotide sequence shown in SEQ ID NO: 7, a polymorphic site located at position 112 of the sequence shown in SEQ ID NO: 7, and a polymorphism of T/A;

the SNP molecular marker Lsnp8 had the nucleotide sequence shown in SEQ ID NO: 8, a polymorphic site located at position 112 of the sequence shown in SEQ ID NO: 8, and a polymorphism of T/C;

the SNP molecular marker Lsnp9 had the nucleotide sequence shown in SEQ ID NO: 9, a polymorphic site located at position 93 of the sequence shown in SEQ ID NO: 9, and a polymorphism of C/T; and

the SNP molecular marker Lsnp10 had the nucleotide sequence shown in SEQ ID NO: 10, a polymorphic site located at position 90 of the sequence shown in SEQ ID NO: 10, and a polymorphism of T/C.

(3) Association analysis between 10 Lsnp sites and cotton resistance to Verticillium wilt:

Analysis of the association between genotype and disease index showed that the association between different haplotypes of Lead-SNPs in 10 QTLs and differences in disease index reached an extremely significant level. As shown in FIG. 2, 10 box plots were the classification statistical analysis between the variation of Lead-SNPs and the disease index in 10 stable QTLs, where the abscissa represented the base types of Lead-SNPs and the ordinate represented the disease index. The disease resistance dominant allele types of Lsnp1 to Lsnp10 were AA, CC, TT, CC, TT, TT, TT, TT, CC, and TT in sequence.

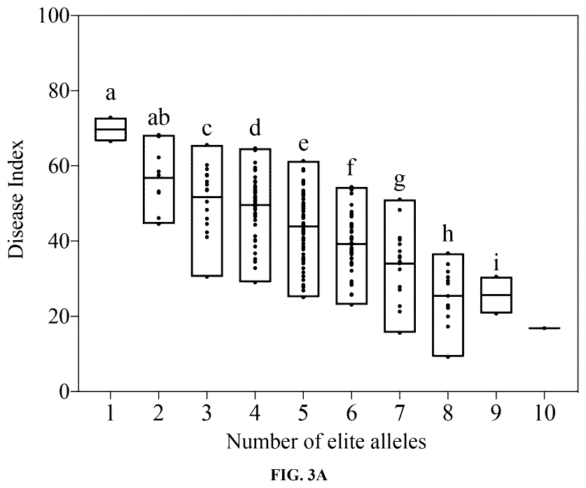

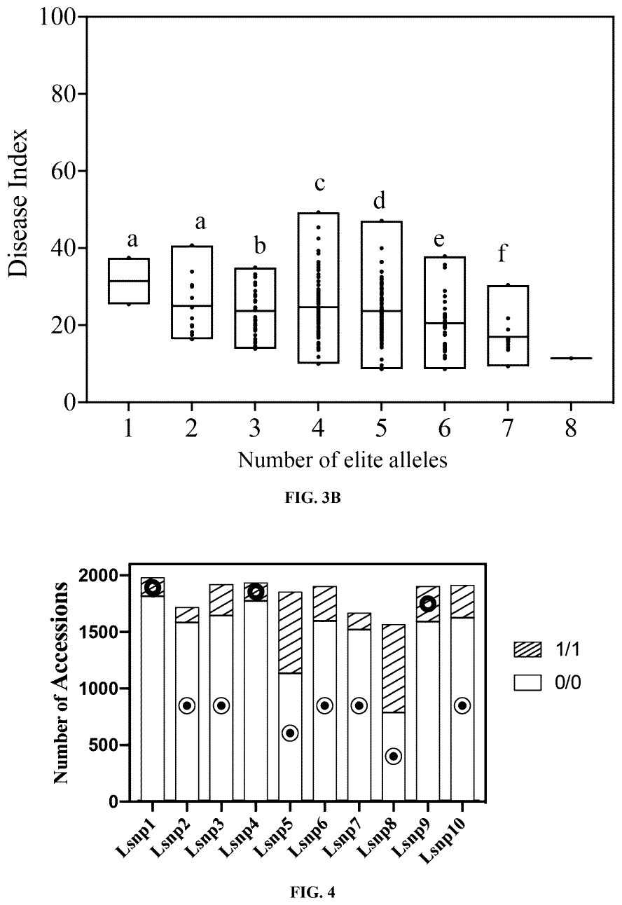

The relationship between the genotype of cotton materials at 10 Lsnp sites and disease resistance in the 290 natural populations was analyzed. The results showed that the more resistant genotypes there were at the 10 Lsnp sites, the lower the disease index of the cotton material was and the stronger the disease resistance was (as shown in FIG. 3A, the 290 natural populations of upland cotton carried at least 1 resistant genotype and at most 10 resistant genotypes, indicating that the number of resistant genotypes carried by cotton materials at the 10 Lsnp sites was positively associated with disease resistance).

### Example 2

The physical locations of 10 Lsnp sites were determined based on a genome of Gossypium hirsutum (AD1) ‘TM-1’ genome ZJU-improved_v2.1_a1 version in a standard line Texas Marker-1 of the upland cotton; where

physical location information of the 10 SNP molecular markers was as follows:

| QTL name | SNP name | Chromosome | Physical location | SNP type |
|---|---|---|---|---|
| qVWR.A01.1 | Lsnp1 | A01 | 111929609 | A/G |
| qVWR.A01.2 | Lsnp2 | A01 | 117983536 | C/T |
| qVWR.A07.2 | Lsnp3 | A07 | 90971603 | T/A |
| qVWR.A10.1 | Lsnp4 | A10 | 108893925 | C/T |
| qVWR.A11.2 | Lsnp5 | A11 | 119799615 | T/C |
| qVWR.A13.1 | Lsnp6 | A13 | 105434827 | T/A |
| qVWR.D01.1 | Lsnp7 | D01 | 1696495 | T/A |
| qVWR.D07.1 | Lsnp8 | D07 | 13371656 | T/C |
| qVWR.D08.2 | Lsnp9 | D08 | 61445110 | C/T |
| qVWR.D10.1 | Lsnp10 | D10 | 22542365 | T/C |

### Example 3

In order to further objectively evaluate the accuracy of the 10 Verticillium wilt resistance SNP markers in Example 1, a natural population of 419 materials for Verticillium wilt resistance association analysis of upland cotton published by other teams was used, referring to: Ma et al. High-quality genome assembly and re-sequencing of modern cotton cultivars provide resources for crop improvement. Nat Genet, 2021, 53:1385-1391. The same analysis was conducted on the 10 Lsnp sites, and it was found that 9 SNPs except Lsnp2 could be detected in the population of 419 cotton materials, and all of them were resistant genotypes at Lsnp2. The more materials in this population had resistant genotypes at the 10 Lsnp sites, the lower the disease index of the cotton material was and the stronger the disease resistance was (as shown in FIG. 3B, the 419 natural populations of upland cotton carried at least 2 resistant genotypes and at most 9 resistant genotypes, indicating that the number of resistant genotypes carried by cotton materials at the 10 Lsnp sites was positively associated with disease resistance).

The above results showed that the 10 Lsnp sites detected through multi-year multi-point disease resistance identification and association analysis were closely related to the Verticillium wilt resistance in cotton. The number of resistant genotypes carried by cotton materials at the 10 Lsnp sites was positively associated with disease resistance, which could be verified in different natural populations. By collecting the genotype data of 2,033 Chinese varieties with publicly available re-sequencing data, the resistant/susceptible genotype frequencies of the 10 Lsnp sites in Chinese varieties were analyzed. It was found that among the 10 Lsnp sites, the major alleles were all TM-1 type, and Lsnp1, Lsnp4, and Lsnp9 were susceptible alleles in the vast majority of Chinese varieties and had great potential for use in future breeding for Verticillium wilt resistance.

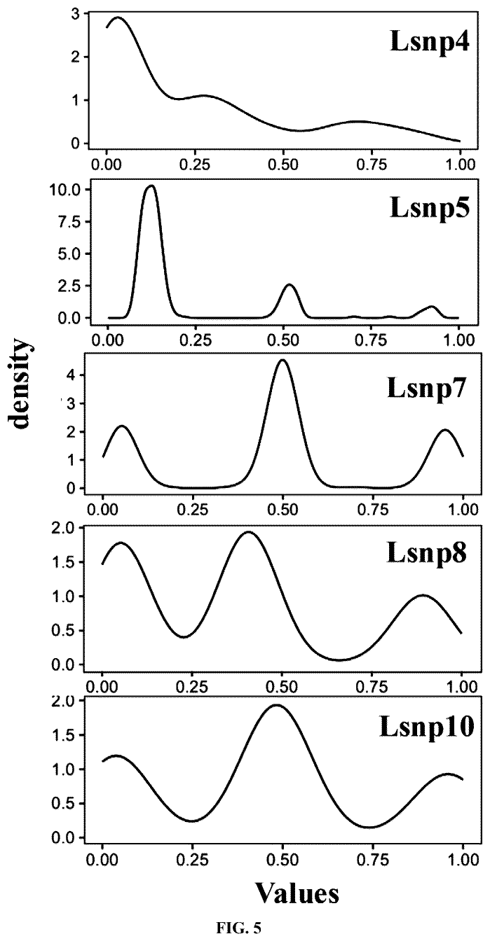

As shown in FIG. 4: there were 2033 cotton varieties used for statistics, where ordinate represented the number of varieties and abscissa represented the 10 Lsnp sites. 0/0 and 1/1 represented the same base type as the upland cotton standard line Texas Marker-1 and variant base type, respectively. The type containing a circle represented the resistant allele types, among which Lsnp1, Lsnp4, and Lsnp9 were rare resistant gene loci.

The genome re-sequencing data of the 2,033 Chinese varieties came from the following five research papers:

He et al. The genomic basis of geographic differentiation and fiber improvement in cultivated cotton. Nat Genet, 2021, 53:916-924;

Li et al. Cotton pan-genome retrieves the lost sequences and genes during domestication and selection. Genome Biol, 2021a, 22:119;

Li et al. Genomic analyses reveal the genetic basis of early maturity and identification of loci and candidate genes in upland cotton (Gossypium hirsutum L.). Plant Biotechnol J, 2021b, 19:109-123;

Li et al. Combined GWAS and eQTL analysis uncovers a genetic regulatory network orchestrating the initiation of secondary cell wall development in cotton. New Phytol, 2020, 226:1738-1752;

Ma et al. Resequencing a core collection of upland cotton identifies genomic variation and loci influencing fiber quality and yield. Nat Genet, 2018, 50:803-813.

### Example 4

(1) Design of sequencing primer set based on 10 Lsnp sites:

First, 500 bp DNA sequences before and after 10 Lsnp sites were extracted for primer design to ensure that the DNA sequence where the Lsnp was located could be specifically amplified. The primers were optimized to obtain 10 sequencing primer sets, and the primer sequences were shown in SEQ ID NO: 11 to SEQ ID NO: 30 in sequence.

Since this application link hoped to provide a high-throughput detection method for multiple samples, a method of using barcode to label multiple samples and combining same with next-generation sequencing was developed. The core idea of barcode and sequencing in this application link was derived from the patent “A method, kit, and use for genotyping using high-throughput sequencing (202010523088.0)”, which disclosed a method for genotyping by high-throughput sequencing. According to the description of the patent, based on the 10 primer sets that could specifically amplify the DNA sequence where Lsnp was located, an 18 bp linker sequence 5′ATAGCGACGCGTTTCAAC3′ was uniformly added to a 5′ end of the reverse primer in G-Lsnp series primers. The subsequent construction of barcode fragments and amplification of SNP fragments were conducted in accordance with the requirements of this patent (202010523088.0).

(2) Genotyping of 272 individual plants in the F2 population using a sequencing primer set

In the early stage, the F2 population of a resistant variety “Zhongzhimian 2” and a susceptible variety “Xinluzao 36” had been constructed. Sequencing revealed that there were variations in the 5 Lsnp sites, Lsnp4, Lsnp5, Lsnp7, Lsnp8, and Lsnp10, between the two parents, such that the typing primer set was verified using 272 individuals in the F2 population. F2 plants were planted and DNA was extracted, SNP fragments were amplified for each group using the method in (1), and then overlap extension, product mixing, product purification, and sequencing were conducted according to the description of the patent (202010523088.0). After the sequencing data was returned, the server processed the data, the barcode was split, and the genotype was extracted.

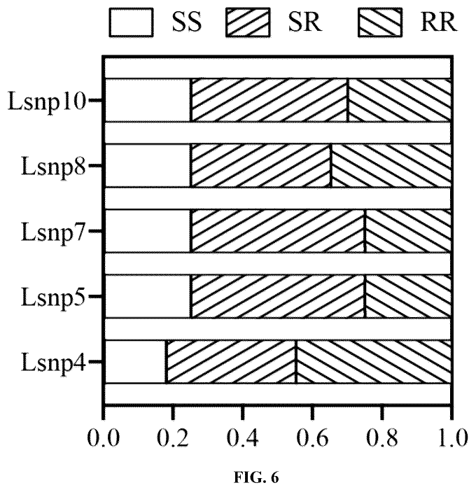

FIG. 5 showed a density plot of the typing results of the read proportions of the resistant allele types at Lsnp4, Lsnp5, Lsnp7, Lsnp8, and Lsnp10 for 272 samples. The abscissa represented the proportion of resistant allele reads, and the ordinate represented the density. The three peaks from left to right represented materials of homozygous susceptible allele, heterozygous allele, and homozygous resistant allele. The 272 individual plants were typed according to the read ratio of the resistant allele type of each material.

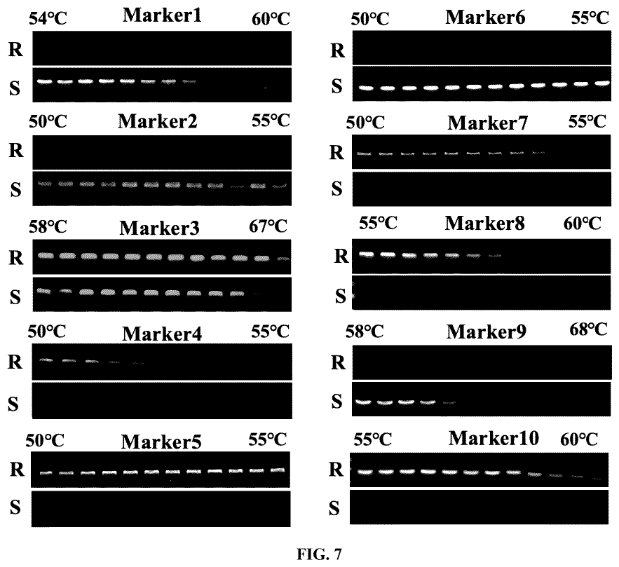

FIG. 6 showed the results of typing 272 individual plants at Lsnp4, Lsnp5, Lsnp7, Lsnp8, and Lsnp10 according to the peak pattern of FIG. 5, where SS represented the homozygous susceptible allele, RR represented the homozygous resistant allele, and SR represented the heterozygous allele. The results showed that among the 272 individuals, 42, 15, 64, 59 and 62 individuals were homozygous resistant genotypes, respectively, while 152, 212, 68, 101 and 80 individuals were homozygous susceptible genotypes, respectively, at the five Lsnp sites of Lsnp4, Lsnp5, Lsnp7, Lsnp8, and Lsnp10.

The results showed that among the 272 individuals, 42, 15, 64, 59 and 62 individuals were homozygous resistant genotypes, respectively, while 152, 212, 68, 101 and 80 individuals were homozygous susceptible genotypes, respectively, at the five Lsnp sites of Lsnp4, Lsnp5, Lsnp7, Lsnp8 and Lsnp10. There were 22 individuals that were homozygous at all five Lsnp sites, with 13 different combination types. This indicated that the 10 pairs of sequencing primer sets as developed could accurately and rapidly conduct genotype detection on multiple samples.

### Example 5

(1) Design of typing primer set based on 10 Lsnp sites:

Since the 10 Lsnp sites were single nucleotide variations, designing primers with annealing temperature sensitivity based on SNP might have limitations such as strict PCR conditions and unstable results. Therefore, it was preferred to use Indels linked to Lsnp to design the typing primer set. The vcf file obtained in Example 1 was used to calculate the Indels highly linked to Lsnp (R²>0.9) using PLINK, and then the DNA sequences of 400 bp before and after the target mutation were extracted for primer design. The primers and PCR conditions were optimized to finally obtain 10 typing primer sets, and the primer sequences are shown in SEQ ID NO: 31 to SEQ ID NO: 50:

| Primer pair | | | Annealing | | |
|---|---|---|---|---|---|
| name | Primer sequence | Band indication | temperature | | Product |
| M-Lsnp1 | Forward: SEQ ID NO: 31 | Susceptibility | 54-57° | C. | 251 bp |
| | Reverse: SEQ ID NO: 32 | | | | |
| M-Lsnp2 | Forward: SEQ ID NO: 33 | Susceptibility | 50-55° | C. | 101 bp |
| | Reverse: SEQ ID NO: 34 | | | | |
| M-Lsnp3 | Forward: SEQ ID NO: 35 | Resistance | 65-67° | C. | 231 bp |
| | Reverse: SEQ ID NO: 36 | | | | |
| M-Lsnp4 | Forward: SEQ ID NO: 37 | Resistance | 50-52° | C. | 230 bp |
| | Reverse: SEQ ID NO: 38 | | | | |
| M-Lsnp5 | Forward: SEQ ID NO: 39 | Resistance | 50-55° | C. | 343 bp |
| | Reverse: SEQ ID NO: 40 | | | | |
| M-Lsnp6 | Forward: SEQ ID NO: 41 | Susceptibility | 50-55° | C. | 228 bp |
| | Reverse: SEQ ID NO: 42 | | | | |
| M-Lsnp7 | Forward: SEQ ID NO: 43 | Resistance | 50-53.5° | C. | 256 bp |
| | Reverse: SEQ ID NO: 44 | | | | |
| M-Lsnp8 | Forward: SEQ ID NO: 45 | Resistance | 55-57° | C. | 345 bp |
| | Reverse: SEQ ID NO: 46 | | | | |
| M-Lsnp9 | Forward: SEQ ID NO: 47 | Susceptibility | 58-60° | C. | 265 bp |
| | Reverse: SEQ ID NO: 48 | | | | |
| M-Lsnp10 | Forward: SEQ ID NO: 49 | Resistance | 55-58° | C. | 281 bp |

Reverse: SEQ ID NO: 50

(2) 10 typing primer sets for PCR and band type detection using resistant/susceptible materials

The genomic DNA of the resistant material “Zhongzhimian 2” and the susceptible material “Xinluzao 36” was extracted by CTAB method. Primers were synthesized according to the 10 pairs of primer sequences according to claim 3, and the PCR program was set according to the annealing temperature specified in (1), a cycle was set to 34×. Next, gel electrophoresis imaging was conducted.

FIG. 7 showed that Marker 1-Marker 10 were typing primer pairs designed based on 10 Lsnp sites, where R represented resistant materials and S represented susceptible materials; above the R rows of the 10 panels was the annealing temperature, and different lanes represented the electrophoresis results within the set annealing temperature. M-Lsnp1, M-Lsnp2, M-Lsnp6, and M-Lsnp9 could specifically amplify bands in susceptible materials at the set annealing temperature, but could not amplify bands in resistant materials; whereas M-Lsnp3, M-Lsnp4, M-Lsnp5, M-Lsnp7, M-Lsnp8, and M-Lsnp10 could specifically amplify bands in resistant materials at the set annealing temperature, but could not amplify bands in susceptible materials.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1.-3. (canceled)

4. A method for detecting Verticillium wilt resistance in upland cotton, comprising: determining disease resistance of the upland cotton by detecting genotypes of the SNP molecular marker combination; wherein the SNP molecular marker combination comprises 10 SNP molecular markers from Lsnp1 to Lsnp10;
wherein the SNP molecular marker Lsnp1 has the nucleotide sequence shown in SEQ ID NO: 1, a polymorphic site located at position 66 of the sequence shown in SEQ ID NO: 1, and a polymorphism of A/G;
the SNP molecular marker Lsnp2 has the nucleotide sequence shown in SEQ ID NO: 2, a polymorphic site located at position 144 of the sequence shown in SEQ ID NO: 2, and a polymorphism of C/T;
the SNP molecular marker Lsnp3 has the nucleotide sequence shown in SEQ ID NO: 3, a polymorphic site located at position 67 of the sequence shown in SEQ ID NO: 3, and a polymorphism of T/A;
the SNP molecular marker Lsnp4 has the nucleotide sequence shown in SEQ ID NO: 4, a polymorphic site located at position 127 of the sequence shown in SEQ ID NO: 4, and a polymorphism of C/T;
the SNP molecular marker Lsnp5 has the nucleotide sequence shown in SEQ ID NO: 5, a polymorphic site located at position 126 of the sequence shown in SEQ ID NO: 5, and a polymorphism of T/C;
the SNP molecular marker Lsnp6 has the nucleotide sequence shown in SEQ ID NO: 6, a polymorphic site located at position 125 of the sequence shown in SEQ ID NO: 6, and a polymorphism of T/A;
the SNP molecular marker Lsnp7 has the nucleotide sequence shown in SEQ ID NO: 7, a polymorphic site located at position 112 of the sequence shown in SEQ ID NO: 7, and a polymorphism of T/A;
the SNP molecular marker Lsnp8 has the nucleotide sequence shown in SEQ ID NO: 8, a polymorphic site located at position 112 of the sequence shown in SEQ ID NO: 8, and a polymorphism of T/C;
the SNP molecular marker Lsnp9 has the nucleotide sequence shown in SEQ ID NO: 9, a polymorphic site located at position 93 of the sequence shown in SEQ ID NO: 9, and a polymorphism of C/T; and
the SNP molecular marker Lsnp10 has the nucleotide sequence shown in SEQ ID NO: 10, a polymorphic site located at position 90 of the sequence shown in SEQ ID NO: 10, and a polymorphism of T/C;
physical location information of the 10 SNP molecular markers is as follows:

| QTL name | SNP name | Chromosome | Physical location | SNP type |
|---|---|---|---|---|
| qVWR.A01.1 | Lsnp1 | A01 | 111929609 | A/G |
| qVWR.A01.2 | Lsnp2 | A01 | 117983536 | C/T |
| qVWR.A07.2 | Lsnp3 | A07 | 90971603 | T/A |
| qVWR.A10.1 | Lsnp4 | A10 | 108893925 | C/T |
| qVWR.A11.2 | Lsnp5 | A11 | 119799615 | T/C |
| qVWR.A13.1 | Lsnp6 | A13 | 105434827 | T/A |
| qVWR.D01.1 | Lsnp7 | D01 | 1696495 | T/A |
| qVWR.D07.1 | Lsnp8 | D07 | 13371656 | T/C |
| qVWR.D08.2 | Lsnp9 | D08 | 61445110 | C/T |
| qVWR.D10.1 | Lsnp10 | D10 | 22542365 | T/C |

the physical location information of the SNP molecular markers is determined based on a genome of Gossypium hirsutum (AD1) ‘TM-1’ genome ZJU-improved_v2.1_a1 version in a standard line Texas Marker-1 of the upland cotton.

5. The method according to claim 4, wherein a criterion for detecting the Verticillium wilt resistance in the upland cotton is that a larger number of resistant genotypes indicates a stronger disease resistance.

6. (canceled)

7. A method for performing early prediction, screening, or breeding of Verticillium wilt resistance in upland cotton comprising:
(1) acquiring a SNP molecular marker combination associated with Verticillium wilt resistance in upland cotton; wherein the SNP molecular marker combination comprises 10 SNP molecular markers from Lsnp1 to Lsnp10;
(2) conducting genome-wide association study of Verticillium wilt resistance in cotton; and
(3) conducting Association analysis between 10 Lsnp sites and cotton resistance to Verticillium wilt and determining a relationship between disease resistance and the number of resistant genotypes at Lsnp1 to Lsnp 10;
wherein in step (1) the SNP molecular marker Lsnp1 has the nucleotide sequence shown in SEQ ID NO: 1, a polymorphic site located at position 66 of the sequence shown in SEQ ID NO: 1, and a polymorphism of A/G;
the SNP molecular marker Lsnp2 has the nucleotide sequence shown in SEQ ID NO: 2, a polymorphic site located at position 144 of the sequence shown in SEQ ID NO: 2, and a polymorphism of C/T;
the SNP molecular marker Lsnp3 has the nucleotide sequence shown in SEQ ID NO: 3, a polymorphic site located at position 67 of the sequence shown in SEQ ID NO: 3, and a polymorphism of T/A;
the SNP molecular marker Lsnp4 has the nucleotide sequence shown in SEQ ID NO: 4, a polymorphic site located at position 127 of the sequence shown in SEQ ID NO: 4, and a polymorphism of C/T;
the SNP molecular marker Lsnp5 has the nucleotide sequence shown in SEQ ID NO: 5, a polymorphic site located at position 126 of the sequence shown in SEQ ID NO: 5, and a polymorphism of T/C;
the SNP molecular marker Lsnp6 has the nucleotide sequence shown in SEQ ID NO: 6, a polymorphic site located at position 125 of the sequence shown in SEQ ID NO: 6, and a polymorphism of T/A;
the SNP molecular marker Lsnp7 has the nucleotide sequence shown in SEQ ID NO: 7, a polymorphic site located at position 112 of the sequence shown in SEQ ID NO: 7, and a polymorphism of T/A;
the SNP molecular marker Lsnp8 has the nucleotide sequence shown in SEQ ID NO: 8, a polymorphic site located at position 112 of the sequence shown in SEQ ID NO: 8, and a polymorphism of T/C;
the SNP molecular marker Lsnp9 has the nucleotide sequence shown in SEQ ID NO: 9, a polymorphic site located at position 93 of the sequence shown in SEQ ID NO: 9, and a polymorphism of C/T; and
the SNP molecular marker Lsnp10 has the nucleotide sequence shown in SEQ ID NO: 10, a polymorphic site located at position 90 of the sequence shown in SEQ ID NO: 10, and a polymorphism of T/C.

8. The method according to claim 7, wherein an SNP type is analyzed by sequencing relevant fragments of the SNP molecular markers amplified using the primer set shown in SEQ ID NO: 11 to SEQ ID NO: 30 in sequence.

9. The method according to claim 4, wherein a primer set is used for amplifying nucleotide fragments where the SNP molecular markers Lsnp1 to Lsnp 10 are located, and primer sequences corresponding to the SNP molecular markers Lsnp1 to Lsnp10 in the primer set are shown in SEQ ID NO: 31 to SEQ ID NO: 50 in sequence.

10. The method according to claim 9, wherein a PCR amplification result of a primer set having the primer sequences shown in SEQ ID NO: 31 to SEQ ID NO: 32 shows positive, indicating a susceptible genotype, and an annealing temperature is 54° C. to 57° C.;
a PCR amplification result of a primer set having the primer sequences shown in SEQ ID NO: 33 to SEQ ID NO: 34 shows positive, indicating a susceptible genotype, and an annealing temperature is 50° C. to 55° C.;
a PCR amplification result of a primer set having the primer sequences shown in SEQ ID NO: 35 to SEQ ID NO: 36 shows positive, indicating a resistant genotype, and an annealing temperature is 65° C. to 67° C.;
a PCR amplification result of a primer set having the primer sequences shown in SEQ ID NO: 37 to SEQ ID NO: 38 shows positive, indicating a resistant genotype, and an annealing temperature is 50° C. to 52° C.;
a PCR amplification result of a primer set having the primer sequences shown in SEQ ID NO: 39 to SEQ ID NO: 40 shows positive, indicating a resistant genotype, and an annealing temperature is 50° C. to 55° C.;
a PCR amplification result of a primer set having the primer sequences shown in SEQ ID NO: 41 to SEQ ID NO: 42 shows positive, indicating a susceptible genotype, and an annealing temperature is 50° C. to 55° C.;
a PCR amplification result of a primer set having the primer sequences shown in SEQ ID NO: 43 to SEQ ID NO: 44 shows positive, indicating a resistant genotype, and an annealing temperature is 50° C. to 53.5° C.;
a PCR amplification result of a primer set having the primer sequences shown in SEQ ID NO: 45 to SEQ ID NO: 46 shows positive, indicating a resistant genotype, and an annealing temperature is 55° C. to 57° C.;
a PCR amplification result of a primer set having the primer sequences shown in SEQ ID NO: 47 to SEQ ID NO: 48 shows positive, indicating a susceptible genotype, and an annealing temperature is 58° C. to 60° C.; and
a PCR amplification result of a primer set having the primer sequences shown in SEQ ID NO: 49 to SEQ ID NO: 50 shows positive, indicating a resistant genotype, and an annealing temperature is 55° C. to 58° C.

11. (canceled)

12. (canceled)

13. The method according to claim 7, wherein disease resistance dominant allele types of the SNP molecular markers Lsnp1 to Lsnp10 are AA, CC, TT, CC, TT, TT, TT, TT, CC, and TT in sequence.


SEQUENCE LISTING (50 sequences)

SEQ ID NO: 1 - DNA, 211 bp, Gossypium hirsutum
  source 1..211: mol_type = genomic DNA; note = SNP molecular marker Lsnp1
atgtcgctccatctggtgttgaatggcgagggcaacggaataagcaaggtcgatgacgtgcccgtacgacatacaccatagaaagtaggtgtcgtaagtattaacgatgttagtgctctttcgctcccccattaacgtgtgagccaaaatggcgcgtaggtacctcagggacggagggagaaccgatgccttggagcggctaggattgtaa

SEQ ID NO: 2 - DNA, 242 bp, Gossypium hirsutum
  source 1..242: mol_type = genomic DNA; note = SNP molecular marker Lsnp2
cgtaagaatccagcacacagcgaggtaagtctccacgagtaatcagattatgttccgtgcgcagattagggtgccagtcaacacctgctaaaagataatacatttatcaaccagtttagcagatcgtgccttgtgcatgaaaactccaatattctatttcagaaccaataaataaatcattaattaggttcatctttcaaatctgactctatcatatgccaagatcacagttcccatttatt

SEQ ID NO: 3 - DNA, 253 bp, Gossypium hirsutum
  source 1..253: mol_type = genomic DNA; note = SNP molecular marker Lsnp3
attgcgatttcattctaccgaccttcaacccatgttttcatggaaaaagacgagttgatttggtcatgagtttgacatatatccttgtctttgtttactttatatgcgcattgcatggacatgattgttttgttaagctgttcaaaatattccttccgatttgttgatctagaaagatcaatccctgtacccatcttctcttttattcttccatgaattttatttttggtcatatcacattccataataagga

SEQ ID NO: 4 - DNA, 299 bp, Gossypium hirsutum
  source 1..299: mol_type = genomic DNA; note = SNP molecular marker Lsnp4
tagtgtgtcacattgaagtgttttttgaaaaccatgttttcattgaaaacccttcttgatgtttagaaacttttaccgtttaaacttgtaaacctttgtagtggcagaaacatgttagtttaaaaacagttaagtatcgaaaaatcaaaccttctttgttatggcttaaaaataatggatactactctccaggtaggattcaagaaaagaggaggtgagtcaattagactgcttaagtacctagctcttccatgatccaatcctagacatgcacacatccattgccacactttaatctt

SEQ ID NO: 5 - DNA, 249 bp, Gossypium hirsutum
  source 1..249: mol_type = genomic DNA; note = SNP molecular marker Lsnp5
cgtgaccttttttcttctcattgttttaggatttagcaaaaaccctaatttcaatagaggatatatggaagaagatattgtagggtttgcttttgaatggagtggttgatgagttgtttaaatagtgaagggaatgaaggttttggagggaatttttgggttaatttttgagaaagatgcatgcaccttacttattaagaaagtccagttgtcaatattttttgaaataaatatggggatacggatggt

SEQ ID NO: 6 - DNA, 229 bp, Gossypium hirsutum
  source 1..229: mol_type = genomic DNA; note = SNP molecular marker Lsnp6
tgtagccacctaaaacaagaaaatgataagtatgttatcatgggtgaagcttgaacagaatcagacatgtttttatgatattcgtcatctcaggttacattgaggattcatccaatgctggtaatttcaaacttagctccagcctcttgttcatacatagtggaaagtaaataaacaattctttctatttcttttttcctccattaacacaaaggataagcaggagagc

SEQ ID NO: 7 - DNA, 308 bp, Gossypium hirsutum
  source 1..308: mol_type = genomic DNA; note = SNP molecular marker Lsnp7
ctattaaaaactggccttatacgtcaacatgaggtacacgtagcacgctacatgtaactatttggttattttgtcaaccacaccaatttttagcagcaaaaatggatgaaatttttaacaggaatgatcaatttgttctttgatctaacgtatagggactaatttgattattttttgagtaaagagggaaaaatgcaatttgactcttaatagaggggtttccatggtacttttaacaatgatgaaaatataatctaagtcaacataggggcatgcggacaaccagataaatgaagttcccacctaag

SEQ ID NO: 8 - DNA, 251 bp, Gossypium hirsutum
  source 1..251: mol_type = genomic DNA; note = SNP molecular marker Lsnp8
aagtaaatgaaatattccgagtctgggattttaaaggaatcgtgccttaacgtattgggtgtgatttattaaatcttggataagtggatgttcttttaaaattttattgtataagtattttggcccaagtcattttggggaaaattagaatgctgtgccctaacgcattgggtgtggtatcttctttctctgaaataataagggtcttaatacgtagccttttaagttttgttaaggattatgtttttttc

SEQ ID NO: 9 - DNA, 246 bp, Gossypium hirsutum
  source 1..246: mol_type = genomic DNA; note = SNP molecular marker Lsnp9
tccgtcgatcccgaacaaacaacacttcttttaaaaaaatttaaaatacctcaacatgcttaaaatattaggctaaatgctcaatttaggtcccaaacttttaggaatttctcacgtaaagactaaatctttcaaaatcatcatataataacccaacttttacaaaagtgatcaaataaaggtttttcacctaattcaactcaatttatagtaaaaattaagtgaatgttagacctgtccaagagt

SEQ ID NO: 10 - DNA, 203 bp, Gossypium hirsutum
  source 1..203: mol_type = genomic DNA; note = SNP molecular marker Lsnp10
ccattgacagggataaaaagttcccatttccacaatttgaacttaaattagcatgtcaatggcattttgcatctaatattatacaacaatgtcaagacacgtgcaaccctatacatttctgtcctaacaaattcatatcgaaacaccaacaggtacgtttataggtccaatgcaaaaatttgtctcctgaaagtctgacgcct

SEQ ID NO: 11 - DNA, 21 bp, synthetic construct
  source 1..21: mol_type = other DNA; note = primer sequence for amplifying SNP molecular marker Lsnp1
atgtcgctccatctggtgttg

SEQ ID NO: 12 - DNA, 21 bp, synthetic construct
  source 1..21: mol_type = other DNA; note = primer sequence for amplifying SNP molecular marker Lsnp1
ttacaatcctagccgctccaa

SEQ ID NO: 13 - DNA, 21 bp, synthetic construct
  source 1..21: mol_type = other DNA; note = primer sequence for amplifying SNP molecular marker Lsnp2
cgtaagaatccagcacacagc

SEQ ID NO: 14 - DNA, 24 bp, synthetic construct
  source 1..24: mol_type = other DNA; note = primer sequence for amplifying SNP molecular marker Lsnp2
aataaatgggaactgtgatcttgg

SEQ ID NO: 15 - DNA, 20 bp, synthetic construct
  source 1..20: mol_type = other DNA; note = primer sequence for amplifying SNP molecular marker Lsnp3
attgcgatttcattctaccg

SEQ ID NO: 16 - DNA, 25 bp, synthetic construct
  source 1..25: mol_type = other DNA; note = primer sequence for amplifying SNP molecular marker Lsnp3
tccttattatggaatgtgatatgac

SEQ ID NO: 17 - DNA, 27 bp, synthetic construct
  source 1..27: mol_type = other DNA; note = primer sequence for amplifying SNP molecular marker Lsnp4
tagtgtgtcacattgaagtgttttttg

SEQ ID NO: 18 - DNA, 24 bp, synthetic construct
  source 1..24: mol_type = other DNA; note = primer sequence for amplifying SNP molecular marker Lsnp4
aagattaaagtgtggcaatggatg

SEQ ID NO: 19 - DNA, 25 bp, synthetic construct
  source 1..25: mol_type = other DNA; note = primer sequence for amplifying SNP molecular marker Lsnp5
cgtgaccttttttcttctcattgtt

SEQ ID NO: 20 - DNA, 24 bp, synthetic construct
  source 1..24: mol_type = other DNA; note = primer sequence for amplifying SNP molecular marker Lsnp5
accatccgtatccccatatttatt

SEQ ID NO: 21 - DNA, 25 bp, synthetic construct
  source 1..25: mol_type = other DNA; note = primer sequence for amplifying SNP molecular marker Lsnp6
tgtagccacctaaaacaagaaaatg

SEQ ID NO: 22 - DNA, 22 bp, synthetic construct
  source 1..22: mol_type = other DNA; note = primer sequence for amplifying SNP molecular marker Lsnp6
gctctcctgcttatcctttgtg

SEQ ID NO: 23 - DNA, 25 bp, synthetic construct
  source 1..25: mol_type = other DNA; note = primer sequence for amplifying SNP molecular marker Lsnp7
ctattaaaaactggccttatacgtc

SEQ ID NO: 24 - DNA, 24 bp, synthetic construct
  source 1..24: mol_type = other DNA; note = primer sequence for amplifying SNP molecular marker Lsnp7
cttaggtgggaacttcatttatct

SEQ ID NO: 25 - DNA, 25 bp, synthetic construct
  source 1..25: mol_type = other DNA; note = primer sequence for amplifying SNP molecular marker Lsnp8
aagtaaatgaaatattccgagtctg

SEQ ID NO: 26 - DNA, 26 bp, synthetic construct
  source 1..26: mol_type = other DNA; note = primer sequence for amplifying SNP molecular marker Lsnp8
gaaaaaaacataatccttaacaaaac

SEQ ID NO: 27 - DNA, 19 bp, synthetic construct
  source 1..19: mol_type = other DNA; note = primer sequence for amplifying SNP molecular marker Lsnp9
tccgtcgatcccgaacaaa

SEQ ID NO: 28 - DNA, 26 bp, synthetic construct
  source 1..26: mol_type = other DNA; note = primer sequence for amplifying SNP molecular marker Lsnp9
actcttggacaggtctaacattcact

SEQ ID NO: 29 - DNA, 23 bp, synthetic construct
  source 1..23: mol_type = other DNA; note = primer sequence for amplifying SNP molecular marker Lsnp10
ccattgacagggataaaaagttc

SEQ ID NO: 30 - DNA, 20 bp, synthetic construct
  source 1..20: mol_type = other DNA; note = primer sequence for amplifying SNP molecular marker Lsnp10
aggcgtcagactttcaggag

SEQ ID NO: 31 - DNA, 20 bp, synthetic construct
  source 1..20: mol_type = other DNA; note = primer set for detecting verticillium wilt resistance in upland cotton
gctattaaaattaaaggccg

SEQ ID NO: 32 - DNA, 20 bp, synthetic construct
  source 1..20: mol_type = other DNA; note = primer set for detecting verticillium wilt resistance in upland cotton
gattggagcattgagtgtga

SEQ ID NO: 33 - DNA, 20 bp, synthetic construct
  source 1..20: mol_type = other DNA; note = primer set for detecting verticillium wilt resistance in upland cotton
actcgatcaagcaccatctg

SEQ ID NO: 34 - DNA, 18 bp, synthetic construct
  source 1..18: mol_type = other DNA; note = primer set for detecting verticillium wilt resistance in upland cotton
ccgtcccaaaggcagtat

SEQ ID NO: 35 - DNA, 21 bp, synthetic construct
  source 1..21: mol_type = other DNA; note = primer set for detecting verticillium wilt resistance in upland cotton
aagacgagttgatttggtcat

SEQ ID NO: 36 - DNA, 20 bp, synthetic construct
  source 1..20: mol_type = other DNA; note = primer set for detecting verticillium wilt resistance in upland cotton
aaactggatttgtctggtgc

SEQ ID NO: 37 - DNA, 24 bp, synthetic construct
  source 1..24: mol_type = other DNA; note = primer set for detecting verticillium wilt resistance in upland cotton
ccgttagtttgaaaacaattagtt

SEQ ID NO: 38 - DNA, 19 bp, synthetic construct
  source 1..19: mol_type = other DNA; note = primer set for detecting verticillium wilt resistance in upland cotton
tatcagggaacgacgcagg

SEQ ID NO: 39 - DNA, 20 bp, synthetic construct
  source 1..20: mol_type = other DNA; note = primer set for detecting verticillium wilt resistance in upland cotton
ttgagtcttgcgtgtctacc

SEQ ID NO: 40 - DNA, 19 bp, synthetic construct
  source 1..19: mol_type = other DNA; note = primer set for detecting verticillium wilt resistance in upland cotton
ggtgctaataccttcctcg

SEQ ID NO: 41 - DNA, 23 bp, synthetic construct
  source 1..23: mol_type = other DNA; note = primer set for detecting verticillium wilt resistance in upland cotton
cggttacaaacacgtaagtaact

SEQ ID NO: 42 - DNA, 22 bp, synthetic construct
  source 1..22: mol_type = other DNA; note = primer set for detecting verticillium wilt resistance in upland cotton
cattttcttgttttaggtggct

SEQ ID NO: 43 - DNA, 23 bp, synthetic construct
  source 1..23: mol_type = other DNA; note = primer set for detecting verticillium wilt resistance in upland cotton
gtgtacataagcagagagacaag

SEQ ID NO: 44 - DNA, 21 bp, synthetic construct
  source 1..21: mol_type = other DNA; note = primer set for detecting verticillium wilt resistance in upland cotton
tgatactccgattactgaggt

SEQ ID NO: 45 - DNA, 27 bp, synthetic construct
  source 1..27: mol_type = other DNA; note = primer set for detecting verticillium wilt resistance in upland cotton
aattatatagcttagccttaaatctaa

SEQ ID NO: 46 - DNA, 18 bp, synthetic construct
  source 1..18: mol_type = other DNA; note = primer set for detecting verticillium wilt resistance in upland cotton
agatgttgagcaagccct

SEQ ID NO: 47 - DNA, 25 bp, synthetic construct
  source 1..25: mol_type = other DNA; note = primer set for detecting verticillium wilt resistance in upland cotton
gacaatatacaaggttaacccagtg

SEQ ID NO: 48 - DNA, 24 bp, synthetic construct
  source 1..24: mol_type = other DNA; note = primer set for detecting verticillium wilt resistance in upland cotton
ttgtcttagaaatgccttgatacc

SEQ ID NO: 49 - DNA, 26 bp, synthetic construct
  source 1..26: mol_type = other DNA; note = primer set for detecting verticillium wilt resistance in upland cotton
tcgatctaaatctcatgcataattag

SEQ ID NO: 50 - DNA, 20 bp, synthetic construct
  source 1..20: mol_type = other DNA; note = primer set for detecting verticillium wilt resistance in upland cotton
ccccacggaatgaagaagat